(19) United States
(12) Patent Application Publication
(10) Pub. No.: US 2008/0097296 A1
(43) Pub. Date: Apr. 24, 2008

(54) Removable hub assembly for medical device

(75) Inventor: Henry Pepin, Loretto, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: Boston Scientific Scimed, Inc.

(21) Appl. No.: 11/505,121

(22) Filed: Aug. 16, 2006

(51) Int. Cl. A61M 25/00

(52) U.S. Cl. 604/103

(57) ABSTRACT

A removable hub and/or manifold structure, assembly, and methods, and medical devices including such hub and/or manifold structures. For example, a hub and/or manifold assembly that is adapted and configured to be removably attached to a shaft of an elongated medical device. The hub assembly can include a hub having a proximal portion and a distal portion, and a mechanical connection structure including a first body portion and a second body portion. The first and second body portions can be removably fastenable together about the distal portion of the hub and the proximal portion of a shaft to connect the hub to the shaft. Elongated medical devices including such a removable hub assembly and methods of attaching a removable hub assembly to a medical device are also disclosed.

### FIELD OF THE INVENTION

The invention relates to medical devices, for example, elongated medical devices for intracorporal use including a hub and/or manifold assembly. More particularly, the invention relates to a selectively removable hub assembly for use on an elongated intracorporal medical device.

### BACKGROUND

The use of intracorporal medical devices, such as intravascular catheters, guidewires, or the like, has become an effective method for treating many types of disease. For example, in some treatments, an intracopreal device is inserted into the anatomy, such as the vascular system, of the patient and navigated to a desired target site, and can be used in treating the target site. Using this method, many target sites in the patient's anatomy can be accessed, including the coronary, cerebral, and peripheral vasculature. Examples of therapeutic purposes for intravascular devices include percutaneous transluminal angioplasty (PTA) and percutaneous transluminal coronary angioplasty (PTCA).

Many medical devices, such as catheters, include a hub and/or manifold at the proximal end to facilitate manipulation of the catheter during navigation within the anatomy, and/or to interface with other devices. For example, the hub may act as a grip through which a physician may urge and/or navigate the catheter by applying longitudinal and/or torsional forces to the proximal portion of the catheter. Additionally, the hub may be adapted to interface with other devices (e.g., inflation device, guide wire, fluid delivery device, etc.) used during a procedure.

A number of different catheters including a variety of hub and/or manifold structures, assemblies, and methods are known, each having certain advantages and disadvantages. However, there is an ongoing need to provide alternative hub and/or manifold structures, assemblies, and methods.

### SUMMARY

Some embodiments of the invention relate to alternative hub and/or manifold structures, assemblies, and methods, and medical devices including them. For example, some embodiments relate to a hub and/or manifold assembly that is adapted and configured to be removably attached to a shaft of an elongated medical device. One example embodiment includes a removable hub assembly for attachment to a proximal portion of a shaft of an elongated medical device. The hub assembly can include a hub having a proximal portion and a distal portion, and a mechanical connection structure including a first body portion and a second body portion. The first and second body portions can be removably fastenable together about the distal portion of the hub and the proximal portion of a shaft to connect the hub to the shaft. Some embodiments relate to an elongated medical device including such a removable hub assembly. Additionally, some embodiments relate to a method of attaching a hub assembly to a shaft of an elongated medical device. For example, one example method involves providing a medical device shaft including a proximal portion and a distal portion, providing a hub having a proximal portion and a distal portion, providing a mechanical connection structure including a first body portion and a second body portion; aligning the proximal portion of the shaft with the distal portion of the hub; and fastening the first and second body portions together about the distal portion of the hub and the proximal portion of a shaft to attach the hub to the shaft.

The above summary of some embodiments is not intended to describe each disclosed embodiment or every implementation of the present invention. The Figures and Detailed Description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a plan view of an example catheter including a removable hub assembly;

FIG. 2 is a partial longitudinal cross sectional exploded view of the proximal portion of the catheter and the hub assembly of FIG. 1;

FIG. 3 longitudinal cross-sectional view of the proximal portion of the catheter of FIG. 1 showing the hub assembly disposed on the catheter shaft;

FIG. 4 is a transverse cross-sectional view of one example embodiment of a mechanical connection structure;

FIG. 5 is a transverse cross-sectional view of another example embodiment of a mechanical connection structure showing alternative mating and/or locking structures;

FIG. 6 is a transverse cross-sectional view of another example embodiment of a mechanical connection structure showing alternative mating and/or locking structures;

FIG. 7 is a transverse cross-sectional view of another example embodiment of a mechanical connection structure showing alternative mating and/or locking structures;

FIG. 8 is a transverse cross-sectional view of another example embodiment of a mechanical connection structure showing alternative mating and/or locking structures and a hinged configuration to the connection structure;

FIG. 9 is a longitudinal cross-sectional view of the proximal portion of a catheter showing another example embodiment of a hub assembly disposed on the catheter shaft; and

FIG. 10 is a longitudinal cross-sectional view of the proximal portion of a catheter showing another example embodiment of a hub assembly disposed on the catheter shaft.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein like reference numerals indicate like elements throughout the several views. The detailed description and drawings illustrate example embodiments of the claimed invention, and are not intended to be limiting.

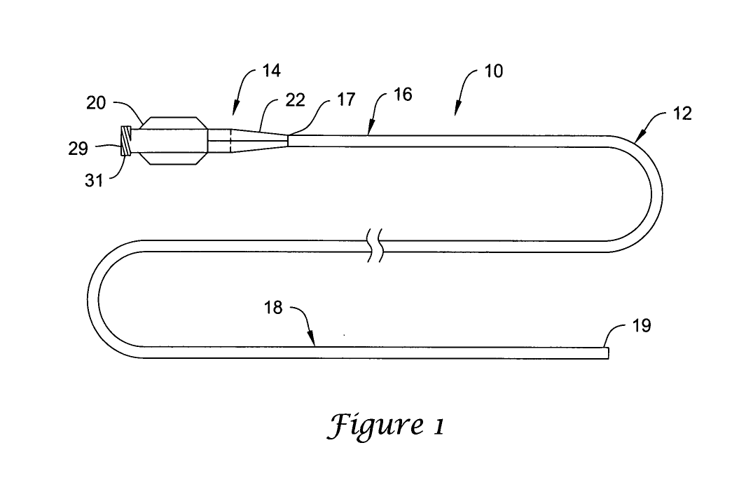

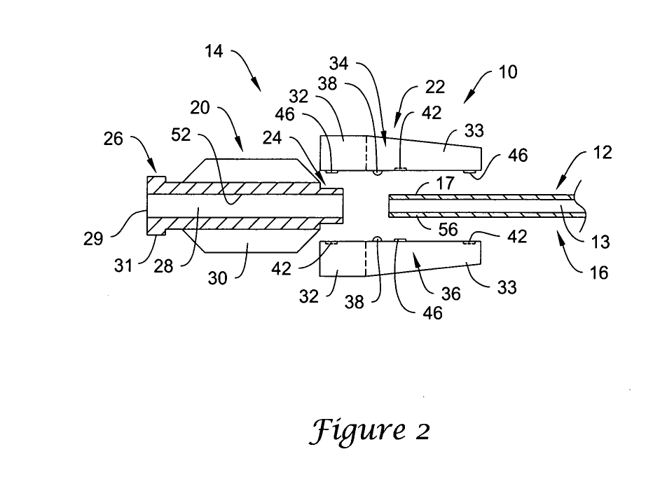

FIG. 1 is a plan view of an example medical device, such as a guide catheter 10 or the like. Catheter 10 includes a shaft 12 having a proximal region 16, a proximal end 17, a distal region 18, and a distal end 19, and may include a lumen 13 (FIG. 2) extending there through. The shaft 12 can include any of a wide variety or structures, layers, and/or materials that may be adapted for the particular usage intended for the catheter, some examples of which are described further below.

A selectively removable hub and/or manifold assembly 14 is disposed adjacent proximal region 16, and as shown, is disposed on and/or about the proximal end 17 of the shaft 12. The hub assembly 14 includes a hub 20, and a mechanical connector structure 22 that is adapted and/or configured to aid in connecting and/or securing the hub assembly 14 to the shaft 12, as will be discussed in more detail below.

Refer now to FIG. 2, which shows the proximal end 17 of the shaft 12, and an exploded view of the hub assembly 14 prior to attachment to the shaft 12. The hub 20 includes a body having a distal portion 24 and a proximal portion 26, and can define one or more lumens, such as lumen 28 extending there through. The lumen 28 may define an inner surface 52 of the hub 20. The hub 22 may include one or more ports, such as port 29 in fluid communication with the lumen 28. The port 29 and lumen 28 may define a pathway through the hub 20. The pathway may, for example, allow for a medical device, such as a guidewire or the like, to extend into the shaft 12. Additionally and/or alternatively, the pathway may provide a path for fluid to enter the shaft 12, such as a contrast medium, medicaments, saline, an inflation fluid, or the like. In some embodiments, the hub 20 may include a plurality of ports in fluid communication with one or more lumens defined therein. For example, plurality of ports may be provided that are in fluid communication with the lumen 28 and/or with additional lumens that may be defined within the shaft 12. For example, the hub 20 may include a Y-type configuration including a device port for insertion of a device into the lumen 28, and a fluid port for insertion of fluids into the lumen 28, or the fluid port may be in communication with a separate lumen. Those of skill in the art and others should understand that a wide variety of hub configurations may be used.

The hub 20 may include a hemostatic or other non-return valve. Additionally, and/or alternatively, the hub 20 may include structure adapted and/or configured to allow for the connection of the hub 20 to other structures and/or devices, such as a Luer fitting, a valve, such as a hemostatic valve, a sealing device, an inflation and/or fluid delivery device, or other fittings, valves, devices, of the like. The fitting, valve, device, or the like may also in turn be adapted for connection to other devices, such as a fluid delivery device and/or may be adapted to allow an additional device, such as a guidewire, to pass there through. For example, in the embodiment shown in FIG. 1, fittings such as threads 31 may be provided on a protrusion that extends about the port 29 for threadable connection to Luer fittings, of the like. Such fittings may be adapted to allow a guidewire, or other such device, to extend and/or be advanced there through in a sealing arrangement, and/or may be adapted for connection to a fluid delivery device for delivery of fluid to the lumen 28. It should also be understood that rather than threads 31, other connecting structures may be used, such as one or more flange, bayonet, or other connector means, or the like.

In yet other embodiments, there may not be a need for the hub 20 to include such a pathway. For example, it is contemplated that the hub assembly 14 may be mounted on elongated medical devices, such as guidewires, embolic protection devices, endoscopes, or the like, where delivery of fluid or other devices to the shaft through the hub 20 is not necessary, but the hub assembly 14 may be desired for manipulation and/or navigation purposes. In some such embodiments, for example, the lumen 28 may extend only partially through the distal portion of the hub 20 adjacent the distal end, for receiving a portion of the shaft 12, or may not include a lumen, but does not necessarily include a fluid pathway through the hub from the shaft 12.

The hub 20 may also include an outer surface that includes structure and/or is configured to allow for gripping and/or manipulation of the hub 20. For example, the hub 20 may include structure that may aid in facilitating manipulation of the catheter 10 during navigation within the anatomy. For example, the hub 20 may include grips 30, such as wings, protrusions, widened portions having any of a wide variety or geometries, or the like, that may aid the physician in gripping and/or manipulating the hub 20 when the physician urges and/or navigates the catheter 12 by applying longitudinal and/or torsional forces to the hub 20.

Referring to FIG. 2, the hub assembly 14 includes a mechanical connection structure 22 that is adapted and/or configured to aid in selectively connecting and/or securing the hub assembly 14 to the shaft 12. The connection structure 22 includes a first body portion 34 and a second body portion 36. The first and second portions 34/36 are configured to matingly engage each other in a locking fashion about a portion of the shaft 12 and a portion of the hub 14 to selectively attach the hub 14 to the shaft 12. For example, the first and second portions 34/36 of the mechanical connection structure 22 can each include a proximal portion 32 and a distal portion 33. The proximal portions 32 can be configured to be disposed over and mate with the distal portion 24 of the hub 20, and the distal portions 33 can be configured to be disposed over and mate with a portion of the shaft 12.

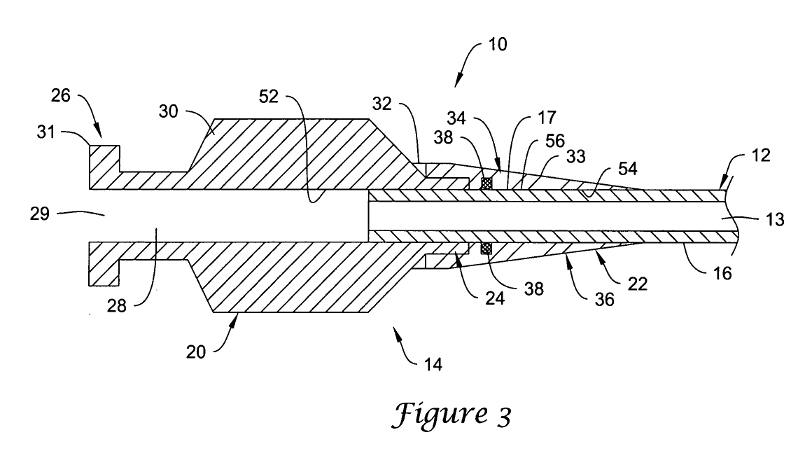

As such, referring now to FIG. 3, the connection structure 22 includes a portion 32 that overlaps with and engages the hub 20, and a portion 33 that overlaps with and engages the shaft 12. For example, when the first and second body portions 34/36 are mated together about the distal portion 24 of the hub 20 and the proximal portion 17 of the shaft 12, the proximal portions 32 may apply a sufficient compressive force to the outer surface of the hub 20 to maintain the connection structure 22 on the hub 20, and the distal portions 33 may apply a sufficient compressive force to the outer surface 56 of the shaft 12 to maintain the connection structure 22 on the shaft 12. In some embodiments, the proximal end 17 of the shaft may be disposed in the lumen 28 of the hub 20 when the connection is made. For example, the outer diameter of the proximal portion of the shaft may be sized such that the outer surface 56 of the shaft 12 is disposed and/or mates with the inner surface 52 of the lumen 28. In other embodiments, however, the proximal end of the shaft 12 may be aligned with, or may be disposed distally of the distal end of the hub 20.

In some embodiments, the proximal portions 32 define a lumen or opening within the connection structure 22 that is configured and/or sized to receive the distal portion of the hub, and apply a compressive force to the outer surface of the hub 20 when the first and second portions 34/36 are mated together about the hub 20. For example, the lumen or opening defined by the proximal portions 32 can be sized slightly or somewhat smaller than the outer diameter of the distal portion of the hub, such that when the first and second portions 34/36 are mated together about the hub 20, a compressive force is generated onto the surface of the hub 20 by the inner surface of the first and second portions 34/36. Such a compressive force may, for example, create frictional engagement between the hub and the connection structure.

Similarly, the distal portions 33 may define a lumen or opening within the connection structure 22 that is configured and/or sized to receive the proximal portion of the shaft 12, and apply a compressive force to the outer surface of the shaft 12 when the first and second portions 34/36 are mated together about the shaft 12. For example, the lumen or opening defined by the distal portions 33 can be sized slightly or somewhat smaller than the outer diameter of the proximal portion of the shaft 12, such that when the first and second portions 34/36 are mated together about the shaft 12, a compressive force is generated onto the surface of the shaft 12 by the inner surface of the first and second portions 34/36. Such a compressive force may, for example, create frictional engagement between the shaft 12 and the connection structure 22. As such, by providing a connection between the connection structure 22 and each of the hub 20 and the shaft 12, the connection structure 22 can be used to connect the shaft 12 to the hub 20. In some embodiments, a secure, fluid-tight connection can be achieved between the catheter shaft 12 and the hub 20.

In some embodiments, the mechanical connection structure 22 can also be configured to function as a strain relief between the shaft 12 and the hub 20. For example, the mechanical connection structure 22 may include structure and/or material that provide for a transition in flexibility characteristics between that of the hub 20 and the shaft 12, and may ease the transition from catheter shaft 12 to hub 20. For example, the distal portions 33 may be tapered, and/or include other structure and/or material that would provide it with a progressive and/or stepwise change in flexibility in a distal direction, and provide structural support for shaft 12. By being adapted to function in this manner, the mechanical connection structure 22 can help to avoid kinking the shaft 12 at the junction of the shaft 12 and the hub 20.

The first and second body portions 34/36 of the connection structure 22 may include structure that allow them to matingly engage and fasten together about the hub 20 and/or shaft 12 to connect the shaft 12 to the hub 20 in a suitable manner. For example, one or both of the body portions 34/36 may include one or more structures, such as one or more protrusion, lip, flange, outcropping, overhang, protuberance, extension, projection, latch, hasp, tab, or the like that is adapted to mate with another such structure and/or a corresponding groove, channel, opening, aperture, or cavity disposed on or within the other of the one or both of the body portions 34/36. For example the first and second body portions 34/36 may include one or more protrusions 46 that are adapted to engage and/or mate with a corresponding aperture 42 defined on the other of the first and second body portions 34/36 to provide for a selective locking engagement of the two portions 34/36. The protrusions 46 and corresponding apertures 42 can be configured, for example to mate in a press-fit and/or snap-fit type configuration to selectively lock the two portions 34/36 together about the shaft 12 and/or hub 20. For example, to lock the two portions 34/36 together, they may be configured such that the two portions 34/36 need only to be aligned, and pressed together to engage the one or more protrusions 46 with the corresponding apertures 42 to lock the two portions 34/36 together. The connection structure 22 and/or the locking structures, such as the protrusions 46 and/or apertures 42, may have sufficient structural integrity to provide a robust connection. It should be understood to those of skill in the art and others, that any of a wide variety of alternative structures and/or components may be used to allow the body portions 34/36 to matingly engage and be fasten together about the hub 20 and/or shaft 12 in such a matter to connect the shaft 12 to the hub 20.

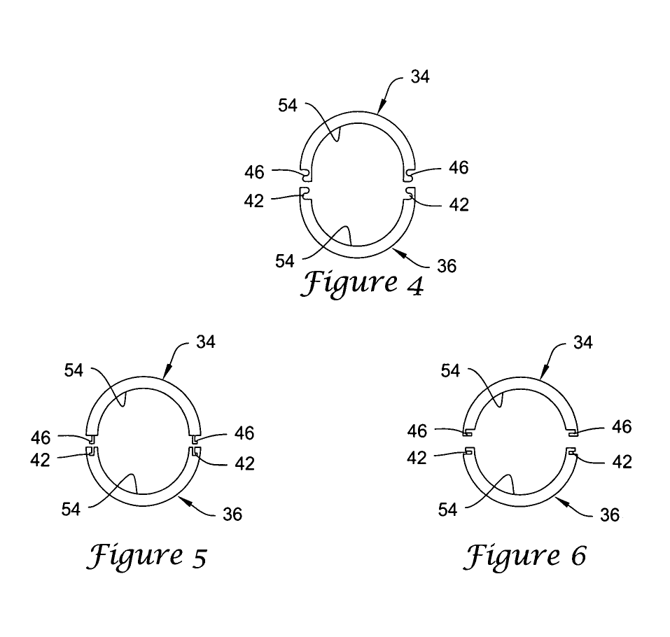

For example, refer now to FIG. 4 which shows a cross-sectional view of one example of a connection structure 22 including protrusions 46 and corresponding apertures 42 that may be characterized, for example, as including a tongue and groove type arrangement. For example, the each of the first and second body portions 34/36 may include an aperture 42 that may be characterized as a groove extending there along, and each of the first and second body portions 34/36 also includes a protrusion 46 extending there along that can be configured to matingly engage the groove of the other body portion when the two body portions are pressed together. As can be appreciated, the protrusions 46 may be slightly deformable such that they can extend about the opposing protrusion when pressed together, but may be sufficiently elastic to recover and extend into the opposing groove when pressed into position.

For another example, refer to FIG. 5, which shows a cross-sectional view of another example of a connection structure 22 including protrusions 46 and corresponding apertures 42 that may be characterized, for example, as including a tab and slot type arrangement. For example, the protrusions 46 may be generally curved and/or L-shaped tabs that are adapted to mate with and extend within generally curved and/or L-shaped slots of the other body portion when the two body portions are pressed together. Again, the protrusions 46 may be sufficiently deformable such that they can be pressed into the slots when the two body portions are pressed together, but may be sufficiently elastic to recover and extend into the slots when pressed into position.

For another example, refer to FIG. 6, which shows a cross-sectional view of another example of a connection structure 22 including protrusions 46 and corresponding apertures 42 that may be characterized, for example, as including a tongue and groove type arrangement similar in some respects to the embodiment of FIG. 4, but including generally squared protrusions 46 and grooves 42, rather than the generally rounded shapes shown in FIG. 4.

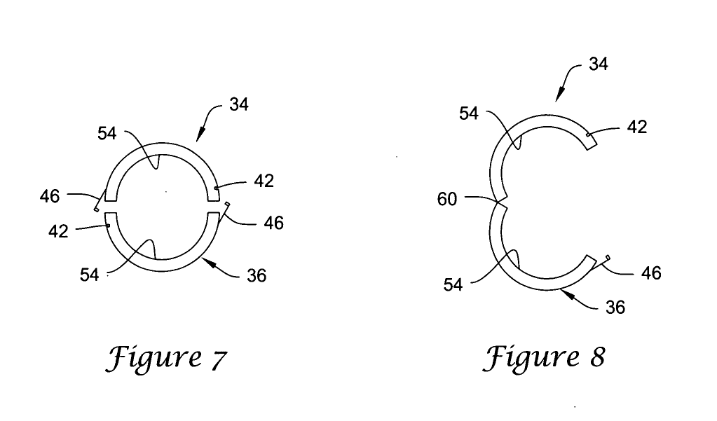

For another example, refer to FIG. 7, which shows a cross-sectional view of another example of a connection structure 22 including protrusions 46 and corresponding apertures 42 that may be characterized, for example, as including a latch and groove type arrangement. For example, the protrusions 46 may be generally latch like members that are adapted to mate with and extend within a slot 42 of the other body portion when the two body portions are pressed together, and the latches are pushed into the slots.

In the embodiments shown thus far, the first and second portions 34/36 are completely separate members that can mate together. However, this is not necessary in all embodiments. For example, in some embodiments, the first and second portions 34/36 may be connected, for example hingedly and/or pivotally connected, or the like. For example, refer to FIG. 8, where the first and second portions 34/36 are hingedly connected at connection point and/or hinge 60. As such, the connection member 22 may be a single member including two or more body portions connected together in a manner that allows them to have an open configuration, as shown in FIG. 8, but also include a closed and/or locked position, for example, when they are disposed about the hub 20 and/or shaft 12.

As indicated above, the first and second portions 34/36 may be selectively and/or releasable disposed about the hub 20 and shaft 12 to selectively and/or releasable connect the hub 20 to the shaft 12. In that regard, connections structures may be configured to be releasable, when desired. In some embodiments, this may entail simply allowing and/or configuring the portions 34/36 such that they may be taken apart, when desired. For example, in a snap-fit and/or press fit type arrangement, the connecting structure, such as the protrusions 46 and grooves 42, may be configured that they can lockingly engage to fasten the portions 34/36 together, but also configured such that when a predetermined and/or sufficient force is applied, they can be pried or otherwise disengaged from one another. In some embodiments, this may be achieved by a user applying sufficient force by hand to deform and/or detach the connections structure, while in other embodiments, a release mechanism and/or tool (not shown) can be provided to disengage the portions 34/36, allowing the connection structure 22 to be unfastened and removed from the shaft 12 and/or hub 20.

In some, but not necessarily all embodiments, the selectively detachable hub assembly 14 may provide for certain advantages. For example, unlike hub assemblies that are permanently connected to a shaft of a catheter, with a removable hub assembly 14, it may be possible for a user to attach the hub to the shaft in a configuration that is desirable to the particular user. For example, a user may align the wings of the hub with the catheter to be in a desirable orientation with one or more curves that may be present in the shaft 12. Additionally, the ability to remove the hub assembly 14 from the shaft 12 allows the physician to place a larger device, such as a larger guide catheter or stiffening device over the outside of the catheter. Additionally, multiple configurations of hubs may be usable with a single catheter shaft and/or multiple configurations of catheter shafts 12 may be usable with a single hub. Further, hub assemblies 14 the catheter shafts 12 can be manufactured separately, and thereafter, the hub can be attached to the catheter when desired. This can eliminate some manufacturing problems associated with permanently attaching a hub to a shaft. These and other potential advantages that may be provided in some embodiments will be appreciated by those of skill in the art an others.

In some embodiments, the connection structure 22 and/or the hub 20 and/or shaft 12 may include additional structure to aid in making the connection. For example, referring back to FIGS. 2 and 3, one or more compression structures, such as compression members and/or structures 38 may be disposed on the inner surface of one or both of the first and second portions 34/36. In some embodiments, the compression fitting 38 may be a pliant, resilient, or compressible member or layer on the inner surface of either or both the proximal and/or distal portions of the connection structure 22. In the embodiment shown, compression members 38 are disposed on the inner surface of the distal portions 33 of the connection structure 22, and may be configured to engage and compress against the outer surface of the shaft 12, for example to provide for better connection and/or a fluid tight seal, when the connection structure 22 is mated about the shaft 12. In other embodiments, such compression members may also be disposed on the inner surface of the proximal portions 32 of the connection structure 22, and may be configured to engage and compress against the outer surface of the hub 20, for example to provide for better connection and/or a fluid tight seal, when the connection structure is mated about the hub 20. It should also be understood that additional and/or alternative compression structures may be used. In some embodiments, the compression members 38 can function by deforming to fit the geometry of the shaft 12 and/or hub 20.

In the embodiment shown, the compression fittings 38 can involve a single discrete area such as a band defined on the inner surface of the connection structure 22, or may involve multiple discrete areas along the connection structure 22. In another embodiment, the entire inner surface of the connection structure 22 may be adapted to function as the compression fitting 38. For example, the entire inner surface of the connection structure 22, or the entire connection structure 22 for that matter, may be made of a material that may be compressible. In other embodiments, however, the compression fitting 38 may be substantially rigid relative to a substantially pliable and/or compressible catheter shaft 12 and/or hub 20. The compression fitting 38 may be configured to provide adequate compression to achieve a fluid-tight connection between the connection structure 22 and the shaft 12 and/or hub 20, but to avoid damage to the shaft 12 and/or hub 20. It will be recognized by one of skill in the art that the suitable amount of compression will vary depending on the composition and/or structure of the shaft 12 and/or hub 20. For example, shafts 12 and/or hubs 20 made of more pliant and compressible material may require less compressive force from the compression fitting 38 to achieve a fluid-tight connection. In yet other embodiments, a compression fitting may be disposed on the outer surface of the shaft 12 and/or hub 20, or both.

In addition, the connection structure 22 and/or the hub 20 and/or shaft 12 may include additional and/or alternative structure to aid in making the connection. For example, the connection structure 22 and/or the hub 20 and/or shaft 12 may include geometries that may matingly engage each other to provide for a better connection. For example, the proximal regions 32 of mechanical connection structure 22 and distal end 24 of hub 20 may have mechanically interlockable geometries, such as one or more retaining members and/or structures, such as one or more teeth, ridges, detents, slots, grooves, protrusions, or any other geometry suitable for interlocking two members. The interlocking geometry on the inside of the proximal region 32 of mechanical connection structure 22 may mate with a corresponding geometry on the outside of the distal end 24 of hub 20 and aid in locking the mechanical connection structure 22 and hub 20 together. Similarly, the distal regions 33 of mechanical connection structure 22 and proximal end 17 of the shaft 17 may also include such mechanically interlockable geometries.

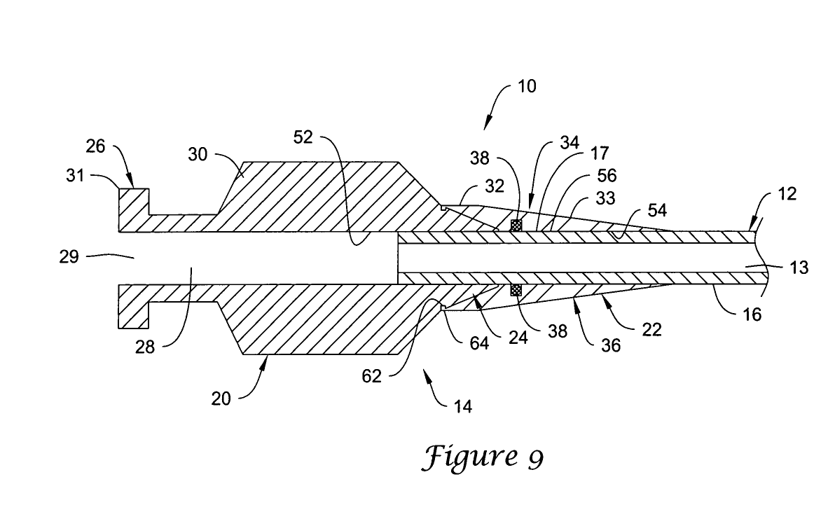

For example, refer now to FIG. 9, which a cross sectional view of a proximal end of another example of a catheter 10 similar to the catheter discussed above, wherein like reference numbers can indicate similar structure. In this embodiments, however, the distal portion 24 of the hub 20 and the proximal portions 32 of the connecting structure 22 include mating geometries that may provide for a better locking engagement of the hub 20 to the connecting structure 22. For example, the hub 20 can include a groove 62 defined therein, and the connecting structure 22 can include a mating protrusion 64 that can be configured to extend within the groove 62. Such an arrangement may provide for better lateral locking and/or fastening of the hub 20 to the connecting structure.

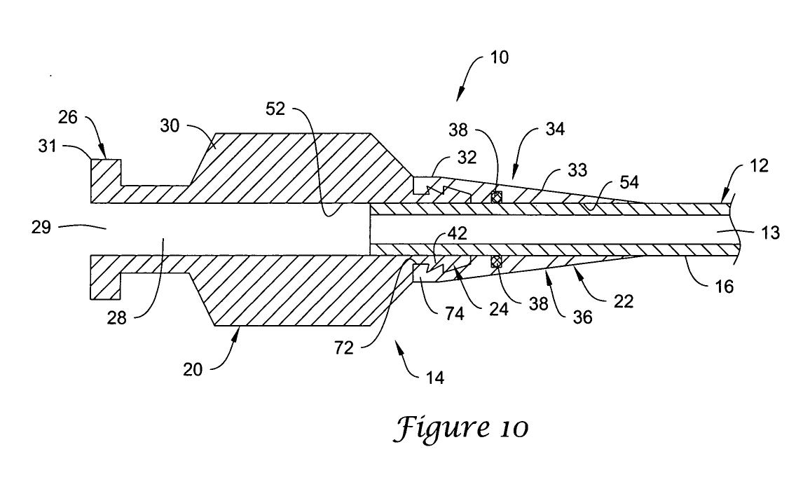

Refer now to FIG. 10, which shows a cross sectional view of a proximal end of another example of a catheter 10 similar to the catheters discussed above, wherein like reference numbers can indicate similar structure. In this embodiments, the distal portion 24 of the hub 20 and the proximal portions 32 of the connecting structure 22 also include mating geometries that may provide for a better locking engagement of the hub 20 to the connecting structure 22. In this embodiment, the hub 20 can include a protrusion 72 including a plurality of projections and/or teeth defined therein, and the connecting structure 22 can also include a mating protrusion 74 also including a plurality of projections and/or teeth defined therein that are adapted to mate with the protrusion 72 on the hub 20. Again, such an arrangement may provide for better lateral locking and/or fastening of the hub 20 to the connecting structure. It should be understood that similar and/or alternative mating structures may also be defined in and/or between the connecting structure 22 and the shaft 12, if so desired.

The hub assembly, and/or the hub 20 and/or connector structure 22 may be made of any suitable materials, for example similar to other typical hub assemblies. For example the hub 20 and/or connector structure 22 may be made from a polymeric material, such as polyamide, polycarbonates, polyether block amide, polyurethane, polyvinylchloride, polypropylene, polyethylene, and the like, or any other suitable material.

The catheter shaft 12 can be manufactured, include structure, be made of materials so as to provide the desired characteristics of the catheter 10, depending upon the intended use. In some embodiments, the shaft 12 and/or the materials and/or structure used to make the shaft 12 can be used to help attach mechanical connection structure 22 to shaft 12 by providing a substrate that is compressible to achieve a compressive connection. Additionally, the shaft 12 can be manufactured using structure and materials so as to maintain a desired level of flexibility and torquability appropriate for maneuvering the catheter 10 as desired, for example, through the vasculature of a patient. In some embodiments, the catheter 10 can include a shaft 12 that is generally characterized as having a tubular member construction that includes at least a single lumen 13 (FIG. 2) extending the length of shaft 12. The lumen 13 within the shaft 12 can possess an inner diameter capable of transmitting fluids, or in some cases, receiving another medical device, such as a guidewire or another catheter, for example, a diagnostic catheter, a balloon catheter, a stent delivery catheter, or the like. In some embodiments, the lumen within shaft 12 is adapted and configured to accommodate another medical device having outer diameters in the range of 5F-10F.

The shaft 12 can be made of a single component or layer, or may have one or more additional layers. For example, in some embodiments, the shaft 12 can have one, two, three, or more layers creating the tubular construction. These layers may change or be constant along the length of the shaft 12. The use of multiple different layers may allow for providing certain desirable characteristics to the shaft 12. For example, one or more of the layers can be made up of one or more tubular segments disposed on or within the shaft and made of suitable material and having suitable structure to impart the desired characteristics to portions of the shaft 12. For example, in some embodiments, an inner layer can be made of a lubricious material to allow for easy insertion of other medical devices. One or more layer may be a reinforcing layer, such as a braid or a coil, adapted to provide desirable characteristics, such as flexibility and/or stiffness characteristics to portions of the shaft 12. For another example, one layer may be made up of a plurality of tubular segments disposed along at least portions of the length of the shaft 12, each segment being made of materials having different durameters to impart varying degrees of flexibility to different sections of the shaft.

The shaft 12 can be constructed using any appropriate technique, for example, by extrusion, a heat bonding process, molding, and the like. Some other examples of suitable catheter shaft constructions and materials can be found in U.S. Pat. Nos. 5,569,218; 5,603,705; 5,674,208; 5,680,873; 5,733,248; 5,853,400; 5,860,963; and 5,911,715, all of which are incorporated herein by reference.

The catheter shaft 12 can be curved or shaped as desired. For example, catheters, such as guide catheters, can include a variety of shapes specific for different bodily passages and procedures. The stabilization of a catheter's position within a patient's anatomy is often achieved through curves or bends imparted into shaft 12. These pre-formed curves act by anchoring a selected portion of shaft 12 against an opposing wall within a patient's vasculature or other body portion. Proper anchoring is often achieved by matching the predisposed shape of the curved shaft 12 with the general curved anatomical shape around a targeted site. In vascular procedures involving treatment to one of the coronary arteries, often a curve is imparted proximate the distal portion of shaft 12 with the intention of placing the catheter's distal tip at a desired angle. In embodiments of catheter 10 that are designed for a procedure in a coronary artery, for example, shaft 12 can be shaped so that when it is inserted through the aorta of the patient, the curvature of shaft 12 will place distal tip at an angle that engages one of the coronary ostia. Those of skill in the art recognize some different shapes by names such as Judkins Right, Judkins Left, Amplatz Right, Amplatz Left, Bentson, Shepherd Hook, Cobra, Headhunter, Sidewinder, Newton, Sones and others, each formed in a different shape.

The catheter shaft 12 and/or components thereof may be manufactured from a number of different materials. For example, catheter shaft 12 may be made of metals, metal alloys, polymers, metal-polymer composites or any other suitable materials. Some examples of suitable metals and metal alloys include stainless steel, such as 300 series stainless steel (including 304V, 304L, and 316L); 400 series martensitic stainless steel; tool steel; nickel-titanium alloy such as linear-elastic or super-elastic Nitinol, nickel-chromium alloy, nickel-chromium-iron alloy, cobalt alloy, tungsten or tungsten alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si), hastelloy, monel 400, inconel 625, or the like; or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polybutylene terephthalate (PBT), polyether block ester, polyurethane, polypropylene (PP), polyvinylchloride (PVC), polyether-ester (for example a polyether-ester elastomer such as ARNITEL® available from DSM Engineering Plastics), polyester (for example a polyester elastomer such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), silicones, polyethylene (PE), Marlex high-density polyethylene, Marlex low-density polyethylene, linear low density polyethylene (for example REXELL®), polyethylene terephthalate (PET), polyetheretherketone (PEEK), polyimide (PI), polyetherimide (PEI), polyphenylene sulfide (PPS), polyphenylene oxide (PPO), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like. In some embodiments shaft 12, or any other portion of catheter 10, can be blended with a liquid crystal polymer (LCP). Of course, any other polymer or other suitable material including ceramics may be used without departing from the spirit of the invention. The materials used to manufacture shaft 12 may also be used for manufacturing other components of catheter 10.

While several of the embodiments explained herein are explained in terms of a hub and/or manifold for use with a catheter, such as a guide catheter, it should be understood that these embodiments are merely illustrative. For example, the several embodiments may be applied to any of a broad variety of medical catheters or devices that may generally include a hub assembly. For example, some or all embodiments may be applied to other types of medical catheters or devices, such as balloon catheters, fluid delivery or infusion catheters, stent delivery catheters, diagnostic catheters, angiographic catheters, atherectomy catheters, billiary catheters, urinary catheters, guidewires, embolic protection devices, endoscopes, occluders, dilators, introducer sheaths and the like, as well as for use in applications in the vasculature, digestive tract, soft tissues, and for other devices adapted for introduction into a body. In some embodiments, the device may be used, for example, in fluidic systems, for providing improved hub-fluidic tube connections, or in electronic or optic systems for connecting a line to a hub, interconnection device, receiver or emitter. It should be understood that such applications are not limited to medical operations on a human patient, and many of these embodiments have additional medical utility, for example, in veterinary applications or for other technologies.

It should be understood that this disclosure is, in many respects, only illustrative. Changes can be made in details, particularly in matters of shape, size, and arrangement of steps without exceeding the scope of the invention. The invention's scope is, of course, defined in the language in which the appended claims are expressed.

What is claimed is:

1. A removable hub assembly for attachment to a proximal portion of a shaft of an elongated medical device, the hub assembly comprising:
a hub having a proximal portion and a distal portion; and
a mechanical connection structure including a first body portion and a second body portion, the first and second body portions being removably fastenable together about the distal portion of the hub and the proximal portion of a shaft to connect the hub to the shaft.

2. The removable hub assembly of claim 1, wherein the first and second body portions fasten together to apply a compressive force to the distal portion of the hub and the proximal portion of a shaft.

3. The removable hub assembly of claim 1, wherein the first and second body portions fasten together to form a lumen extending through and defining an inner surface within the mechanical connection structure, the lumen being adapted to receive the distal portion of the hub and the proximal portion of a shaft.

4. The removable hub assembly of claim 3, wherein the distal portion of the hub has an outer surface having outer diameter, and the lumen includes a proximal portion having an inner diameter that is the same size or smaller than the outer diameter of the distal portion of the hub such that when the first and second body portions are fasten together, the inner surface of the lumen engages the outer surface of the distal portion of the hub.

5. The removable hub assembly of claim 3, wherein the proximal portion of the shaft an outer surface having outer diameter, and the lumen includes a distal portion having an inner diameter that is the same size or smaller than the outer diameter of the proximal portion of the shaft such that when the first and second body portions are fasten together, the inner surface of the lumen engages the outer surface of the shaft.

6. The removable hub assembly of claim 1, wherein the mechanical connection structure includes a compression fitting configured to form a fluid-tight connection with the shaft.

7. The removable hub assembly of claim 1, wherein the mechanical connection structure includes a compression fitting configured to form a fluid-tight connection with the hub.

8. The removable hub assembly of claim 1, wherein the first and second body portions each includes an inner surface adapted to engage the distal portion of the hub and the proximal portion of the shaft when the first and second body portions are fastened together about the distal portion of the hub and the proximal portion of the shaft.

9. The removable hub assembly of claim 8, further including a compression fitting disposed on the inner surface of the body portions.

10. The removable hub assembly of claim 1, wherein the first and second body portions include one or more fastening structures configured to releasably fasten the first and second body portions together.

11. The removable hub assembly of claim 10, wherein the one or more fastening structures include one or more protrusion disposed on the first body portion, and one or more groove, channel, opening, or aperture, disposed on second body portion, wherein the one or more protrusion is adapted to mate with the one or more groove, channel, opening, or aperture to releasably fasten the first and second body portions together.

12. The removable hub assembly of claim 1, wherein the first and second body portions are configured to be selectively and releasably fastened together in a snap fit manner.

13. The removable hub assembly of claim 1, wherein the mechanical connection structure is configured to selectively attach the hub to the shaft in a fluid-tight connection.

14. The removable hub assembly of claim 1, wherein the mechanical connection structure is configured to provide strain relief between the hub and the shaft.

15. An elongated medical device comprising;
an elongated shaft including a distal portion and a proximal portion;
a selectively removable hub assembly removably attached to the proximal portion of the shaft, the hub assembly including:
a hub including a distal portion; and
a mechanical connection structure including a first body portion and a second body portion removably fastened together about the distal portion of the hub and the proximal portion of a shaft to connect the hub to the shaft.

16. The elongated medical device of claim 15, wherein the first and second body portions fasten together to apply a compressive force to the distal portion of the hub and the proximal portion of a shaft.

17. The elongated medical device of claim 15, wherein the first and second body portions fasten together to form a lumen extending through and defining an inner surface within the mechanical connection structure, the lumen being adapted to receive the distal portion of the hub and the proximal portion of a shaft.

18. The elongated medical device of claim 17, wherein the distal portion of the hub has an outer surface having outer diameter, and the lumen includes a proximal portion having an inner diameter that is the same size or smaller than the outer diameter of the distal portion of the hub such that when the first and second body portions are fasten together, the inner surface of the lumen engages the outer surface of the distal portion of the hub.

19. The elongated medical device of claim 17, wherein the proximal portion of the shaft an outer surface having outer diameter, and the lumen includes a distal portion having an inner diameter that is the same size or smaller than the outer diameter of the proximal portion of the shaft such that when the first and second body portions are fasten together, the inner surface of the lumen engages the outer surface of the shaft.

20. The elongated medical device of claim 15, wherein the mechanical connection structure includes a compression fitting configured to form a fluid-tight connection with the shaft.

21. The elongated medical device of claim 15, wherein the mechanical connection structure includes a compression fitting configured to form a fluid-tight connection with the hub.

22. The elongated medical device of claim 15, wherein the first and second body portions each includes an inner surface adapted to engage the distal portion of the hub and the proximal portion of the shaft when the first and second body portions are fastened together about the distal portion of the hub and the proximal portion of the shaft.

23. The elongated medical device of claim 15, further including a compression fitting disposed on the inner surface of the body portions.

24. The elongated medical device of claim 15, wherein the first and second body portions include one or more fastening structures configured to releasably fasten the first and second body portions together.

25. The elongated medical device of claim 24, wherein the one or more fastening structures include one or more protrusion disposed on the first body portion, and one or more groove, channel, opening, or aperture, disposed on second body portion, wherein the one or more protrusion is adapted to mate with the one or more groove, channel, opening, or aperture to releasably fasten the first and second body portions together.

26. The elongated medical device of claim 15, wherein the first and second body portions are configured to be selectively and releasably fastened together in a snap fit manner.

27. The elongated medical device of claim 15, wherein the mechanical connection structure is configured to selectively attach the hub to the shaft in a fluid-tight connection.

28. The elongated medical device of claim 15, wherein the mechanical connection structure is configured to provide strain relief between the hub and the shaft.

29. The elongated medical device of claim 15, wherein the medical device comprises a catheter.

30. An elongated medical device comprising;
an elongated shaft including a distal portion and a proximal portion;
a selectively removable hub assembly removably attached to the proximal portion of the shaft, the hub assembly including:
a hub including a distal portion; and
means for connecting the distal portion of the hub and the proximal portion of a shaft.

31. A method of attaching a removable hub to a medical device shaft, the method comprising:
providing a medical device shaft including a proximal portion and a distal portion;
providing a hub having a proximal portion and a distal portion;
providing a mechanical connection structure including a first body portion and a second body portion that releasably fasten together;
aligning the proximal portion of the shaft with the distal portion of the hub;
fastening the first and second body portions together about the distal portion of the hub and the proximal portion of a shaft to releasable attach the hub to the shaft.
